(19) United States
(12) Patent Application Publication
(10) Pub. No.: US 2024/0246895 A1
(43) Pub. Date: Jul. 25, 2024

(54) ABATEMENT OF LOW LEVEL METHANE THROUGH THE USE OF CATALYTIC, EARTH-ABUNDANT MATERIALS

(71) Applicant: Massachusetts Institute of Technology, Cambridge, MA (US)

(72) Inventors: Desiree Plata, Concord, MA (US); Rebecca Brenneis, Arlington, VA (US)

(73) Assignee: Massachusetts Institute of Technology, Cambridge, MA (US)

(21) Appl. No.: 18/562,120

(22) PCT Filed: May 19, 2022
(86) PCT No.: PCT/US2022/030025

§ 371 Date: Nov. 17, 2023

Related U.S. Application Data
(60) Provisional application No. 63/190,404, filed on May 19, 2021.

(51) Int. Cl. C07C 45/33; B01D 53/86; B01J 23/72; B01J 23/745; B01J 23/755; B01J 29/072; B01J 29/24; B01J 29/46; B01J 37/08; B01J 37/14; C07C 29/50

(52) U.S. Cl. CPC C07C 45/33; B01D 53/864; B01J 23/72; B01J 23/745; B01J 23/755; B01J 29/072; B01J 29/24; B01J 29/46; B01J 37/08; B01J 37/14; C07C 29/50; B01D 2251/102; B01D 2251/11; B01D 2255/20738; B01D 2255/20753; B01D 2255/20761; B01D 2255/504; B01D 2257/7025; B01D 2258/06

(57) ABSTRACT

A system and method for oxidizing methane can include an environmentally friendly catalyst material that converts methane to an oxidized product at low temperatures and concentrations, for example, under 350° C. at concentrations less than 40% methane, including less than 5% methane.

### PRIORITY CLAIM

This application claims priority to U.S. Provisional Patent Application No. 63/190,404, filed May 19, 2021, which is incorporated by reference in its entirety.

### TECHNICAL FIELD

This invention relates to systems and methods of oxidizing methane.

### BACKGROUND

While carbon dioxide is the greenhouse gas at the center of the global climate conversation, methane is arguably the bigger problem. Methane is more potent and much faster acting, yet critically, no control technology exists that can address methane at most of its sources globally.

Society is not prepared to tackle the unique problem that is rising atmospheric methane concentrations, and solutions are needed now. Methane's atmospheric concentration is roughly 0.4% that of Carbon dioxide, yet has contributed fully 25% as much radiative forcing since preindustrial times. Atmospheric methane concentrations have increased almost twice as fast as CO₂ over this time frame as well. The International Panel on Climate Change has established the metric that methane is 86 and 34 times more potent that CO₂ over a 20- and 100-year time horizon, respectively. See, for example, Myhre, G., D. et al., 2013: Anthropogenic and Natural Radiative Forcing. In: Climate Change 2013: The Physical Science Basis. Contribution of Working Group I to the Fifth Assessment Report of the Intergovernmental Panel on Climate Change Cambridge University Press, Cambridge, United Kingdom and New York, NY, USA, which is incorporated by reference in its entirety. These values reflect the fact that methane is a rapidly acting greenhouse which, mole for mole, contributes 10 times more radiative forcing to the atmosphere in 50 years than carbon dioxide will do in 400. The scientific community agrees that dramatic cuts in greenhouse gas emissions are needed by 2030, a ten-year time horizon, in order to avoid a 2 degree Celsius or greater temperature rise. From the IPCC calculation, over a ten-year horizon, methane is over 100 times more potent than CO₂. The same IPCC report predicts that based on current emissions, methane will contribute more radiative forcing than CO₂ in that time. The world has struggled to begin responding to the threat of global climate change over the last 40 years and we are rapidly running out of time. The need for technological innovation to help contain global methane emission is as imminent as it is profound.

### SUMMARY

In general, a system and method for oxidizing methane can include an environmentally friendly catalyst material that converts methane to an oxidized product at low temperatures and concentrations, for example, under 350° C. at concentrations less than 40% methane, including less than 5% methane.

In one aspect, a method of oxidizing methane can include activating a methane oxidation catalyst at an activation temperature at or below about 450° C. while exposed to an activation gas including less than 100% oxygen for an activation time, and exposing the activated methane oxidation catalyst to a reaction gas mixture including less than 100% methane at a temperature of, or less than about 350° C. in the presence of an oxidative agent including less than 100% oxygen to convert the methane to an oxidized product.

In another aspect, a system for oxidizing methane can include a housing including an activation region for a methane oxidation catalyst that is activated at an activation temperature at or below about 450° C. while exposed to an activation gas including less than 100% oxygen for an activation time, and a conversion region for the activated methane oxidation catalyst to convert a reaction gas mixture including less than 100% methane to an oxidized product at a temperature of, or less than about 350° C. in the presence of an oxidative agent including less than 100% oxygen.

In another aspect, a method of oxidizing methane can include exposing a reaction gas mixture including less than 4% methane in the presence of an oxidative agent including less than 22% oxygen to a copper doped zeolite to convert the methane to an oxidized product. In certain circumstances, the reaction gas mixture including less than 4% methane in the presence of the oxidative agent including less than 22% oxygen is exposed to the copper doped zeolite at a temperature of, or less than about 350° C. In certain circumstances, the oxidative agent can be less than 22% oxygen in an inert gas. In certain circumstances, the oxidative agent can be air. The oxidative agent can include about 20% oxygen.

In certain circumstances, the activation time can be 240 minutes or less, 180 minutes or less, 120 minutes or less, or 90 minutes or less.

In certain circumstances, the activation gas can include less than 80% oxygen, less than 60% oxygen, less than 40% oxygen, or about 20% oxygen. An example of an activation gas including about 20% oxygen is air.

In certain circumstances, the activation gas can include an inert gas. The inert gas can be helium, argon or nitrogen.

In certain circumstances, the activation gas can be air.

In certain circumstances, the activation temperature can be below about 450° C., below about 400° C., below about 350° C., below about 300° C., or below about 250° C.

In certain circumstances, the oxidative agent can include a liquid, for example, hydrogen peroxide, hypohalous acids and equilibrating species, dissolved peroxidases, and other liquid oxidants.

In certain circumstances, the oxidative agent can include a solid, for example, metal cofactors such as iron, silver, manganese, or lead.

In certain circumstances, the oxidative agent can include a gas including oxygen gas.

In certain circumstances, the oxidative agent can include less than 100% oxygen, less than 40% oxygen, less than 30% oxygen, or about 20% oxygen. An example of an oxidative agent including about 20% oxygen is air, which contains approximately 21% oxygen.

In certain circumstances, the reaction gas mixture can include less than 50% methane, less than 20% methane, less than 10% methane, less than 5% methane, less than 1% methane, less than 1000 ppm methane, less than 100 ppm methane, or less than 10 ppm methane.

In certain circumstances, the methane oxidation catalyst can include iron, copper, or nickel. For example, the methane oxidation catalyst can include a copper zeolite, an iron zeolite, or a nickel zeolite, such as copper mordenite.

In certain circumstances, the methane oxidation catalyst can include a metal organic framework, a zeolite or an aerogel.

Other aspects, embodiments, and features will be apparent from the following description, the drawings, and the claims.

### BRIEF DESCRIPTION OF THE DRAWINGS

FIG. 1 depicts a system for oxidizing methane.

FIG. 2 is a schematic depicting a reactor diagram. Gas flow pathways displayed above flow left to right. The MFC array provides a single gas stream of variable concentration which flows through the vertical furnace where the catalyst sits at variable temperature. The coiled loops at the GC injection valve allow simultaneous injection and sample collection. The secondary helium system allows flow from the reactor to be vented during activation without contaminating the injection system or leaving void space.

FIGS. 3A-3B are drawings depicting reaction schematics.

FIGS. 4A-4B are a graphs depicting copper loading of catalyst samples prior to activation. ICPMS analysis was performed on catalyst samples after ion exchanges of varied lengths, from 1 to four days. FIG. 4A represents Cu-ZSM5 and FIG. 4B represents CuMOR.

FIGS. 5A-5B is a series of crystallographic diffraction spectra of activated catalyst. XRD analysis was performed on catalyst samples after activations at 450° C. of varied lengths, from 0 to 180 minutes. FIG. 5A represents Cu-ZSM5 and FIG. 5B represents CuMOR.

FIG. 6 is a graph depicting conversion efficiency as a function of reaction temperature.

FIGS. 7A and 7B are graphs depicting conversion efficiency as a function of activation temperature. FIG. 7A shows activations performed under 100% oxygen. FIG. 7B shows activation performed under 20% Oxygen and 80% Helium.

FIG. 8 is a graph depicting a percentage of catalyst efficiency under variable storage conditions. Time between the activation and reaction were at least 1 hour. Helium only samples were not removed from the reactor during storage time. Dry atmosphere samples were stored in a desiccator at room temperatures and a drying oven at 130° C.

FIG. 9 is a graph depicting conversion efficiency and activation time.

FIG. 10 is a graph depicting conversion efficiency and conversion reaction temperature at different activation temperatures.

FIGS. 11A-11B are graphs depicting conversion efficiency over a range of inputs.

FIG. 12 is a graph depicting conversion efficiency over a range of temperatures.

FIGS. 13A-13B are graphs depicting conversion efficiency and production over a range of methane concentrations.

FIG. 14 is a graph depicting conversion efficiency over time. Long-term activity of the catalyst. Low-level methane (2 ppmv methane in 20% oxygen) was catalytically reacted over 300 h under continuous, isothermal operation at 310° C. (asterisks, following 8 h activation) and a traditional two-step process (circles; 450° C., 30 min activation followed by 200° C. continuous reaction).

FIG. 15 is a graph depicting methane conversion rate increases with the input methane concentration over a range of sub-flarable levels. Methane conversion was tested from 2 ppmv to 2% v/v methane in the presence of 20% oxygen in isothermal operation at 310° C. (30 min initial activation in methane-free gas; asterisks), and following 30 min (filled symbol) and 60 min (open symbol) activations (450° C.) and reaction (200° C.) in 20% oxygen. Each data point collected with the freshly prepared catalyst represents at least 20 methane conversion measurements.

FIG. 16 is a graph depicting conversion efficiency and multiple reactivations.

Other aspects, embodiments, and features will be apparent from the following description, the drawings, and the claims.

### DETAILED DESCRIPTION

In general, a system and method for oxidizing methane can include a methane oxidation catalyst that is activated to create a reactive catalyst that oxidizes methane under relatively mild conditions.

A relatively new catalyst technology has been developed to mimic a methanotrophic enzyme that can convert methane to methanol with high efficiency at low temperatures. Biomimetic copper zeolites have so far been studied as a tool for transporting and storing valuable natural gas, with selectivity for methanol as a primary constraint. However, these reaction conditions limit the conversion efficiency of methane to values below 1% at 200° C. Still, the high efficiency of the methane monooxygenase enzyme suggests the potential for much higher efficiencies if one does not optimize for selectivity. Here copper zeolite catalysts were synthesized using a minimally energy intensive, technically practical procedure and evaluated for catalytic efficiency of methane without controlling for selectivity. Reaction parameters were chosen to closely mimic environmental conditions whenever possible. The catalyst was demonstrated to exhibit 46% efficiency at 200° C. with minimal conversion observed as low as 75° C. Complete conversion was achieved by 300° C. Catalytic activation was completed successfully 150° C. lower than previously explored and at atmospheric oxygen concentrations as opposed to 100% oxygen. All reactions took place at ambient pressure with atmospheric concentrations of methane and oxygen suggesting real world applicability. Materials characterization via ICP-MS and XRD suggest that lengthy synthesis steps do not improve catalyst performance.

A catalytic material to convert low-levels of methane from gaseous streams to non-methane molecules, such as CO₂, methanol, or formaldehyde, or other carbon forms is described herein. The catalytic material can be a copper-doped zeolite. This material can be either supported or unsupported. It can function to convert low and atmospheric levels of methane (e.g., 1 ppm to 5%), a potent greenhouse gas, away from methane into other gases, liquids, or solids with lower global warming potentials. The catalyst functions at relatively low temperatures (ambient to 200° C. typically, but up to 350° C.) and can be activated in air (e.g., 20% O₂ as opposed to 100% O₂) or using liquid oxidants. The activation process is typically driven at modestly high temperatures (e.g., 450° C.).

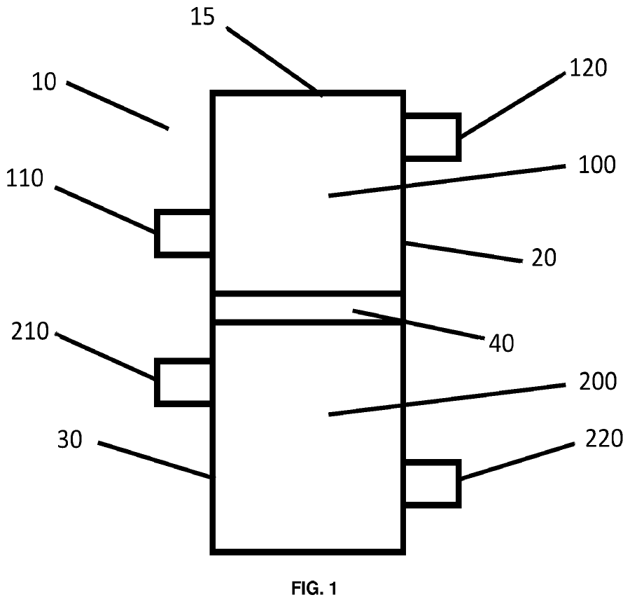

The system for oxidizing methane can include an activation region for a methane oxidation catalyst that is activated at an activation temperature at or below about 450° C. while exposed to an activation gas including less than 100% oxygen for an activation time, and a conversion region for the activated methane oxidation catalyst to convert a reaction gas mixture including less than 100% methane to an oxidized product at a temperature of, or less than about 350° C. in the presence of an oxidative agent including less than 100% oxygen. FIG. 1 depicts a system for oxidizing methane. Referring to FIG. 1, system 10 includes housing 15. Housing 15 includes first chamber 20 and second chamber 30. First chamber 20 and second chamber 30 can be isolated by separator 40. Separator 40 can be a door or removable wall that can allow first chamber 20 and second chamber 30 to be subjected to different conditions. For example, first chamber 20 can include activation region 100 in which a methane oxidation catalyst within the activation region 100 is subjected to the activation temperature and exposed to the activation gas, for the activation time. Activation region 100 can be configured for a first gas flow through first chamber 20 via first chamber inlet 110 and first chamber outlet 120. The first gas flow can include oxygen. Second chamber 30 can include conversion region 200 in which the activated methane oxidation catalyst can be exposed to a reaction gas mixture to convert it to an oxidized product. Conversion region 200 can be configured for a second gas flow through the chamber via second chamber inlet 210 and second chamber outlet 220. The second gas flow can include methane and an oxidative agent.

The system described herein can be used to implement a method of oxidizing methane. The method can include activating a methane oxidation catalyst at an activation temperature at or below about 450° C. while exposed to an activation gas including less than 100% oxygen for an activation time, and exposing the activated methane oxidation catalyst to a reaction gas mixture including less than 100% methane at a temperature of, or less than about 350° C. in the presence of an oxidative agent including less than 100% oxygen to convert the methane to an oxidized product.

Unexpectedly, the system and method described herein can be implemented under relatively mild conditions. For example, the methane oxidation catalyst can be activated at an activation temperature at or below about 450° C. Surprisingly, the activation can take place when exposed to an activation gas including less than 100% oxygen. This contrasts with other systems and methods that require temperatures greater than 450° C. and gas exposure of 100% oxygen to activate a methane oxidation catalysts. Also unexpected is the reactivity of the activated methane oxidation catalyst at relatively low temperatures and under conditions with low methane concentrations and oxygen concentrations. Surprisingly, methane oxidation to an oxidized product, such as methanol, can take place at a temperature of, or less than about 350° C. the presence of less than 100% oxygen gas. More details of these experiments are described below.

In certain circumstances, the methane oxidation catalyst can include iron, copper, or nickel. The methane oxidation catalyst can be a catalyst based on environmentally friendly materials, such as iron or copper. The catalyst can include a support material for the iron, copper or nickel. For example, the methane oxidation catalyst can include a metal organic framework, a zeolite, a clay or an aerogel, which can be the support material. This support material can provide a non-reactive support for the active metal for the methane oxidation catalyst. For example, the methane oxidation catalyst can include a copper zeolite, an iron zeolite, or a nickel zeolite. The zeolite can be an aluminosilicate, such as a pentasil zeolite. For example, the zeolite can be a mordenite or ZSM-5. The methane oxidation catalyst can be made by cation exchange of the support material with the active metal ion, such as the iron, copper, or nickel.

The methane oxidation catalyst can be activated with an activation gas at an activation temperature for an activation time. The activation time can be 12 hours, 10 hours, 8 hours, 6 hours, or less. In certain circumstances, the activation time can be 240 minutes or less, 180 minutes or less, 120 minutes or less, or 90 minutes or less. The system and method described herein can have a short activation time, which allows for rapid reactivation of the methane oxidation catalyst as necessary. In certain circumstances, the activation gas can include an inert gas. The inert gas can be helium, argon or nitrogen, or mixtures thereof. In certain circumstances, the activation gas can be air.

In certain circumstances, the activation gas can include less than 80% oxygen, less than 60% oxygen, less than 40% oxygen, or about 20% oxygen. For example, the activation can take place effectively at ambient oxygen levels, such as the oxygen content in air. This can allow activation to take place without the need for any specialty gases.

In certain circumstances, the activation temperature can be below about 450° C., below about 400° C., below about 350° C., below about 300° C., or below about 250° C. The activation temperature can be as high as 550° C. in the presence of 20% oxygen, which is an unexpected improvement because of the reduced oxygen content required to activate the catalyst.

The oxidation of methane to an oxidized product by the activated methane oxidation catalyst can take place at a temperature of, or less than about 350° C. in the presence of an oxidative agent including less than 100% oxygen. In certain circumstances, the oxidative agent can include a solid, for example, metal cofactors such as iron, silver, manganese or lead. In certain circumstances, the oxidative agent can include a liquid, for example, hydrogen peroxide, hypohalous acids and equilibrating species, dissolved peroxidases, and other liquid oxidants. The oxidative agent can enhance the reactivity of the catalyst. In certain circumstances, the oxidative agent can include a gas including oxygen gas, for example, oxygen in an inert carrier gas or in air. For example, the oxidative agent can include less than 100% oxygen, less than 40% oxygen, less than 30% oxygen, or about 20% oxygen. The oxidative agent can be a combination of one or more of these agents.

In certain circumstances, the system and method can be isothermal. In this example, the temperature is held constant, for example between 250 and 350° C. The activation is accomplished by exposing the methane oxidation catalyst to an oxidative agent in the absence of methane. The exposure can be for an activation period of time of, for example, 10 minutes, 20 minutes, 30 minutes, 40 minutes, 50 minutes, 60 minutes, 90 minutes, or 120 minutes. The activated methane oxidation catalyst can then be exposed to a gas including methane, which is then converted to an oxidation product at the same temperature. In this way, the methane oxidation catalyst is activated (or reactivated) simply by changing the atmosphere the catalyst is exposed to.

Unexpectedly, the system and method described herein can oxidize methane when present in relatively low concentrations. In other systems, methane concentrations of more than 50%, and often 100%, are needed to observe oxidation products. Using the system and method described herein, the reaction gas mixture can include less than 50% methane, less than 20% methane, less than 10% methane, less than 5% methane, less than 1% methane, less than 1000 ppm methane, less than 100 ppm methane, or less than 10 ppm methane. The conversion rate increased over a range of methane concentrations (0.00019-2%), indicating the potential to abate methane from any sub-flammable stream. Oxidation of methane of less than 1000 parts per million concentrations of methane with environmentally friendly catalyst such as a copper or iron zeolite with heat under ambient atmosphere creates opportunities to create systems that have a positive impact on the environment by reducing greenhouse gas impact.

The oxidized products can include methanol, carbon dioxide, and other oxidative products of methane. The conversion efficiency of the methane can be at least 5%, at least 10%, at least 20%, at least 30%, at least 40%, at least 50%, at least 75%, at least 90%, at least 95%, or nearly 100%.

Reducing global CH₄ emissions is a problem complicated by the diversity of its sources and the inertness of its chemical character, rendering it both hard to find and hard to catch. Roughly 40% of major methane production globally comes from non-anthropogenic sources, and yet increases from some of these sources have been observed as consequences of climate warming or other human-induced environmental perturbations. See, for example, The Global Methane Budget 2000-2017, by Marielle Saunois, et al (2020), Earth System Science Data, 12, 1-63, 2020, DOI: 10.5194/essd-12-1561-2020; Walter Anthony, K., et al. Methane emissions proportional to permafrost carbon thawed in Arctic lakes since the 1950s. Nature Geosci 9, 679-682 (2016); Schuur, E., et al. Climate change and the permafrost carbon feedback. Nature 520, 171-179 (2015); Schneider von Deimling, T., et al. (2012). Estimating the near-surface permafrost-carbon feedback on global warming. Biogeosciences, 9, 649-665; Zhang, Z., et al. Wetland methane emissions in future climate change. PNAS 114, (36) 9647-9652; (2017); Neumann, R. B., et al. (2019). Warming effects of spring rainfall increase methane emissions from thawing permafrost. Geophysical Research Letters, 46, 1393-1401; and Walter Anthony, K., et al. 21st-century modeled permafrost carbon emissions accelerated by abrupt thaw beneath lakes. Nat Commun 9, 3262 (2018), each of which is incorporated by reference in its entirety. Unlike man-made sources, it is an engineering challenge to predict where or why these increases will occur. While one can identify sources of anthropogenic methane emissions and how they are likely to behave, more than two thirds of these are diffuse. Diffuse sources occur over large distances at concentrations low enough that trapping the gas physically is unfeasible. Methane itself is nonpolar, poorly acidic, too small for susceptibility by Van der Waals forces, and has a high bond dissociation energy making it highly impervious to chemical interaction. It can participate in redox chemistry, but is only flammable at levels above 5%, and anything below this level cannot be flared. Because atmospheric methane concentration is only 0.000185%, there is a 4 orders of magnitude range of methane concentrations which are a problem for the climate but untouchable by any modern technology. Critically, more than three quarters of global methane emission falls in this category.

Luckily, evolution has a tool for oxidizing methane at ambient temperatures and dilute concentrations. Methane Monooxygenase (MMO) is a metabolic enzyme found in methanotrophic bacteria. It is 100% efficient at converting methane to methanol for use as biomass or energy. See, for example, Murrell, J., et al. Molecular biology and regulation of methane monooxygenase. Arch. Microbiol. 173, 325-332 (2000), which is incorporated by reference in its entirety. This astounding efficiency is the result of high-spin reactive oxygen active sites capable of activating CH bonds, and a redox controlled “breathable” pore structure that efficiently directs flow of relevant gases, preventing errant diffusion that could lead to alternative oxidative pathways. See, for example, Kimberly T. Dinh, et al. Viewpoint on the Partial Oxidation of Methane to Methanol Using Cu- and Fe-Exchanged Zeolites ACS Catalysis 2018 8 (9), 8306-8313, which is incorporated by reference in its entirety. Over the last decade, much progress had been made towards creating a synthetic analog of this advantageous chemistry, and attempts made with transition metal exchanged zeolites have been particularly promising. See, for example, Ambarish R. Kulkarni, et al., Cation-exchanged zeolites for the selective oxidation of methane to methanol Catal. Sci. Technol., 2018; Kimberly T. Dinh, et al. Continuous Partial Oxidation of Methane to Methanol Catalyzed by Diffusion-Paired Copper Dimers in Copper-Exchanged Zeolites J. Am. Chem. Soc. 2019, 141, 11641-11650; B. Ipek and R. F. Lobo Catalytic conversion of methane to methanol on Cu-SSZ-13 using N2O as oxidant Chem. Commun., 2016, 52, 13401; Karthik Narsimhan, et al. Catalytic Oxidation of Methane into Methanol over Copper-Exchanged Zeolites with Oxygen at Low Temperature ACS Cent. Sci. 2016, 2, 424-429; and Yayun Shi, et al. (2020) Quasicatalytic and catalytic selective oxidation of methane to methanol over solid materials: a review on the roles of water, Catalysis Reviews, 62:3, 313-345, each of which is incorporated by reference in its entirety. The precisely designed zeolite pore structural geometries make them useful for enhancing catalytic reactions by forcing metal oxide active sites into unfavorable highly energetic configurations, and squeezing gaseous constituents into contact through tortuous nanoscale channels.

At present, laboratory progress toward this method of methane conversion has been pursued with the goal of transporting and storing valuable extracted methane in mind, with emphasis on maximizing the selectivity of methanol as an oxidative product. Unfortunately, the progress made toward recreating the MMO active site within the zeolite framework has outpaced any technological development to mimic the enzymatic pore gating structure. Without this mechanism, methane to methanol conversion is thermodynamically constrained by the difference in the activation energy between the CH bonds in methane and methanol. See, for example, Allegra A. Latimer, et al. Direct Methane to Methanol: The Selectivity-Conversion Limit and Design Strategies ACS Catal. 2018, 8, 6894-6907, which is incorporated by reference in its entirety. Accumulation of the presence of methanol near or at the active site favors a methanol oxidation side reaction, yielding CO₂.

In order to avoid the pathway of complete oxidation, studies of direct oxidation of methane to methanol at low temperatures have been conducted under highly-engineered laboratory processes that separate gaseous oxygen and methane into sequential steps, and utilize concentrated methane streams to thermodynamically favor methane activation in the presence of methanol. See, for example, Kimberly T. Dinh, et al. Viewpoint on the Partial Oxidation of Methane to Methanol Using Cu- and Fe-Exchanged Zeolites ACS Catalysis 2018 8 (9), 8306-8313; Ambarish R. Kulkarni, et al., Cation-exchanged zeolites for the selective oxidation of methane to methanol Catal. Sci. Technol., 2018; B. Ipek and R. F. Lobo Catalytic conversion of methane to methanol on Cu-SSZ-13 using N₂O as oxidant Chem. Commun., 2016, 52, 13401; Yayun Shi, et al. (2020) Quasicatalytic and catalytic selective oxidation of methane to methanol over solid materials: a review on the roles of water, Catalysis Reviews, 62:3, 313-345; and Allegra A. Latimer, et al., Direct Methane to Methanol: The Selectivity-Conversion Limit and Design Strategies ACS Catal. 2018, 8, 6894-6907, each of which is incorporated by reference in its entirety. This strategy maximizes the selectivity of methanol as the reaction product, yet at such high concentrations of methane, conversion efficiency is often so low that it is not quantified or reported. When it is reported, it is well below 1%. In fact, a theoretical limit of 0.2% has been proposed as the maximum percentage of methane conversion efficiency at which considerable methanol selectivity would still be possible. See, for example, Ambarish R. Kulkarni, et al. Cation-exchanged zeolites for the selective oxidation of methane to methanol Catal. Sci. Technol., 2018; and Allegra A. Latimer, et al., Direct Methane to Methanol: The Selectivity-Conversion Limit and Design Strategies ACS Catal. 2018, 8, 6894-6907, each of which is incorporated by reference in its entirety. As a consequence, the actual conversion capacity of these catalysts has yet to be explored, but the nature of the selectivity values themselves suggest that the reactivity is high even under these controlled conditions. The question becomes, could the catalyst be a useful tool for methane sequestration if the selectivity handicap were removed?

The long-term goal of this project is the synthesis and deployment of a methane sequestration catalyst that is scalable, economically advantageous, and carbon balanced from cradle to grave. It is essential to approach research into methane control measures with scale and timing in mind at all stages, because the time required to bring a concept to market is arguably as important a parameter as its catalytic efficiency. The purpose of this study was two-fold. First, to synthesize a cost effective, low energy, easily reproducible copper zeolite catalyst. Second, to evaluate its catalytic efficiency under environmentally relevant conditions while optimizing for conversion over selectivity.

### Methods

The development of the methodology for this work was an engineering goal in itself. Each objective began with a literature review and survey of existing strategies. Laboratory procedures were chosen to minimize environmentally harsh or energy intensive processes and reaction conditions, to mimic standard environmental conditions as much as possible. Taken together, these methods represent the simplest, most reproducible and environmentally conscious strategy for synthesizing and evaluating copper zeolites and their methane conversion chemistry to date.

### Reactor Design

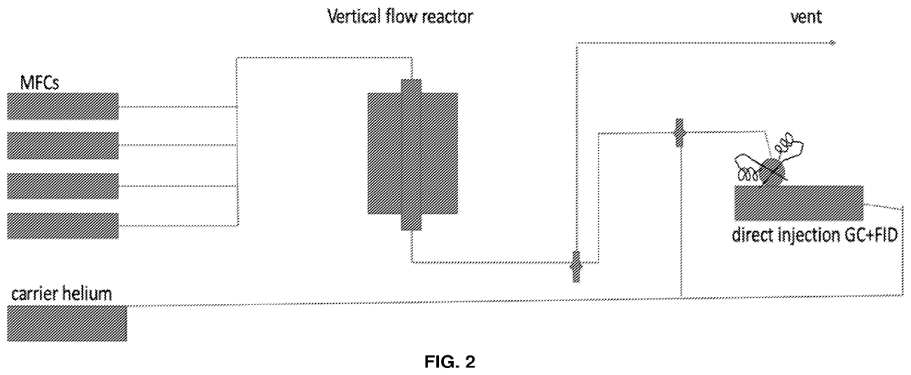

In order to assess the catalyst efficacy in a realistic environment, it was necessary to create a system for analytical gas analysis of the reaction effluent stream. The reactor system used here was designed and optimized for that purpose. Reactions were conducted in a vertical tube furnace (FIG. 2). Gases were delivered by an array of mass flow controllers MFCs connected in series, with software-assisted simultaneous control of four gas constituents at once. Reactor effluent was delivered to an SRI gas chromatograph with flame ionization detector (GC-FID) by direct injection. The GC injection system was supplied by two parallel 5 mL loops controlled by a manual valve, allowing simultaneous sampling and injection. While the GC cannot analyze a continuous gas stream, the sampling valve was used to make frequent repeated injections and collect time series data in real time. GC calibration was performed for CH₄ and CO₂ to deduce an appropriate injection interval that would prevent coelution, in the event that CO₂ was observed. A 10-minute flushing time was established before gases introduced to the reactor were analyzed to allow the effluent to reach steady state concentration values over repeated injections. All data analyzed by this collection method excludes information from the first ten minutes of reaction to avoid this window.

### Catalyst Synthesis

The ion exchange of copper solution with an ammonium-substituted mordenite powder was conducted similar to Narsimhan, K., et al. ACS Cent. Sci. 2016, 2, 424-429, which is incorporated by reference in its entirety. In this study, ion exchange times were shortened to a 24-hour minimum on magnetic stir plates and ambient temperature. Solutions were then vacuum filtered and dried in a laboratory oven at 130° C. for no less than 12 hours and later transferred to a desiccator. This process was shorter, and involved fewer repetitions of ion exchange than frequently observed. No attempt was made to avoid wicking during the drying process, which would require precise control of temperature ramps.

### Standard Reaction Conditions

A standard reaction was formulated that would increase the potential for methane conversion from the “three-step process”, and also bring it closer to conditions where one might need it to perform at methane seeps. Low temperature direct oxidation of methane to methanol over metal zeolite catalyst has mostly been analyzed through a three-step reaction design. See, for example, Yayun Shi, et al. (2020) Quasicatalytic and catalytic selective oxidation of methane to methanol over solid materials: a review on the roles of water, Catalysis Reviews, 62:3, 313-345; and Allegra A. Latimer, et al., Direct Methane to Methanol: The Selectivity-Conversion Limit and Design Strategies ACS Catal. 2018, 8, 6894-6907, each of which is incorporated by reference in its entirety. A high temperature catalyst activation (typically 450 to 800° C.) was followed by a reaction step, wherein the oxidized catalyst is reacted with a high concentration methane stream, often 100%, in the absence of oxygen. This strategy is designed to maximize methane activation while preventing methanol activation. Finally, the catalyst itself was extracted in aqueous solution and analyzed for pore constituents.

Catalyst activation was carried out approximately as in Grudner, S., Chem. Commun., 2016, 52, 2553, which is incorporated by reference in its entirety. In the case where reaction conditions were varied during experimentation, the activation was held constant at the following ambient pressure conditions. Approximately 0.9 grams of copper zeolite (CuMor) was loaded into a vertical tube furnace, and flushed with helium while heating to 450° C., followed by a 30-minute hold at 450° C. the low end of the temperature range explored in the literature, under 100% oxygen. See, for example, Ambarish R. Kulkarni, et al., Cation-exchanged zeolites for the selective oxidation of methane to methanol Catal. Sci. Technol., 2018; Kimberly T. Dinh, et al., Continuous Partial Oxidation of Methane to Methanol Catalyzed by Diffusion-Paired Copper Dimers in Copper-Exchanged Zeolites J. Am. Chem. Soc. 2019, 141, 11641-11650; B. Ipek and R. F. Lobo Catalytic conversion of methane to methanol on Cu-SSZ-13 using N2O as oxidant Chem. Commun., 2016, 52, 13401; Karthik Narsimhan, et al. Catalytic Oxidation of Methane into Methanol over Copper-Exchanged Zeolites with Oxygen at Low Temperature ACS Cent. Sci. 2016, 2, 424-429; Yayun Shi, et al. (2020) Quasicatalytic and catalytic selective oxidation of methane to methanol over solid materials: a review on the roles of water, Catalysis Reviews, 62:3, 313-345; and Allegra A. Latimer, et al., Direct Methane to Methanol: The Selectivity-Conversion Limit and Design Strategies ACS Catal. 2018, 8, 6894-6907, each of which is incorporated by reference in its entirety. The catalyst was then cooled to reaction conditions under helium flush. Catalyst activation has been performed in the literature at temperatures on average between 450 and 850° C., with pressures as high as 6 bar and over time spans ranging from a few hours to a day. These conditions were chosen to be the least intensive possible that would reasonably be expected to establish the reactive oxygen active site.

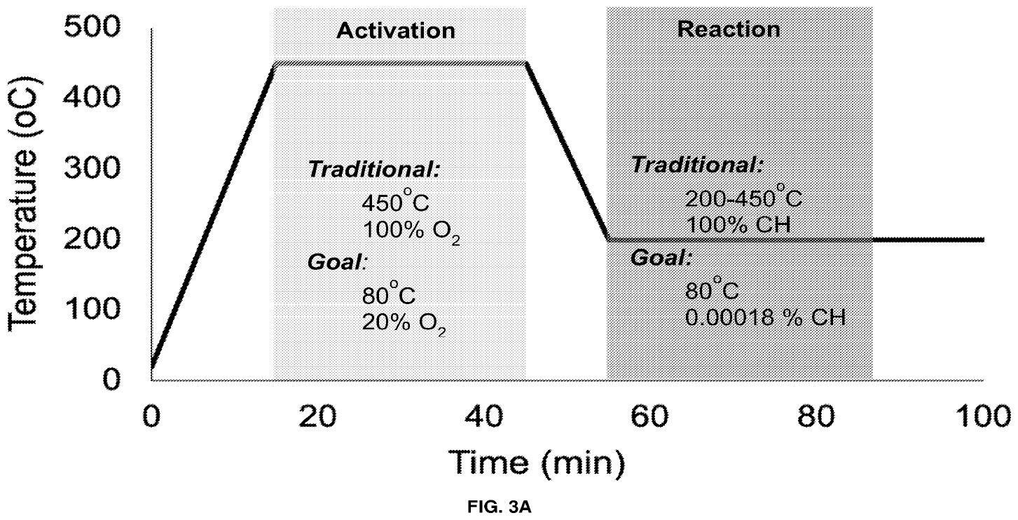

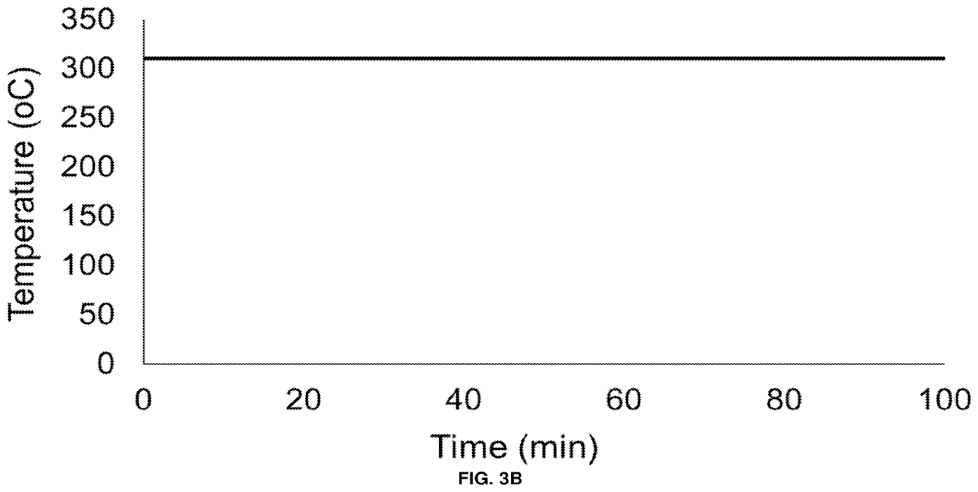

The temperature was then lowered to 200° C. and the feed changed to 1.85 ppm methane, 20% oxygen, with balance helium at flows representing a 30 second residence time in the reaction volume. These concentrations were chosen to closely mimic atmospheric conditions. Overall, this standard reaction (FIG. 3) was designed to parallel an industrial manufacture process, followed by deployment into environments of interest. FIG. 3A shows a standard reaction defined for this study. The two primary steps are an activation to create reactive oxygen species in the reactor and a reaction step to convert methane. Catalyst is flushed with helium between these steps and during temperature transitions. When one parameter is varied all other variables are held in this condition. FIG. 3B shows another approach, where temperature is held constant. This isothermal approach can allow for activation to take place when exposed to an oxidative agent such as oxygen gas in the absence of methane for an activation period of time (for example, 10 minutes, 20 minutes, 30 minutes, 40 minutes, 50 minutes, 60 minutes, 90 minutes, or 120 minutes) and then methane in the presence of an oxidative agent such as oxygen in the conversion or reaction stage.

Conversion efficiency was found by averaging the steady state methane concentration in the reactor effluent between 10 and 30 minutes from the introduction of reaction gases to the reactor. This concentration (C) is then compared with the blank concentration (Co), the result of performing the same experiment in the absence of catalyst. Co values were consistent across temperatures and no combustion was observed. Co values were determined at all temperatures. The standard deviation for all C and Co values were all less than 5% of the mean. A Co was also determined for at 200° C. for with mordenite zeolite powder with no ion exchange and was consistent with the other blank values.

### Materials Characterization

In this study, materials characterization was used to further optimize the efficiency of synthesis procedures. These methods are used in the literature to try to narrowly define a specific active site motif with the aim of engineering the most efficient possible site. Here characterization was conducted on samples of catalyst which had been synthesized with steps of varying duration. The results were designed to be analyzed alongside catalytic efficiency to assess the cost benefit analysis of technical synthesis procedures which may or may not produce an equal increase in methane conversion.

### Inductively Coupled Plasma Mass Spectrometry (ICP-MS)

The weight percent copper loading was analyzed via ICP-MS to determine the effectiveness of the ion exchange procedure. Zeolites are silicate minerals, which cannot be digested for elemental analysis without the use of hydrofluoric acid. To avoid adding this dangerous and resource intensive step to the process, an attempt was made to leach copper out of unoxidized catalyst samples in nitric acid. Cu-ZSM5 was refluxed using a beaker and watch glass in 67% HNO₃ for two hours until the solids were visibly bleached white. The leachate was extracted via pipette, concentrated down to 10% of its initial volume using heat, then reconstituted in 2% HNO₃ for ICP-MS analysis. It is important to note that, due to the nature of the leaching process, not a complete digestion, these values are not a specific quantification of copper values. It is not possible to say what percentage of the copper was successfully leached, however, the method was kept consistent across each sample and if a trend existed the expectation is it would have been detectable. All samples were leached and analyzed in triplicate.

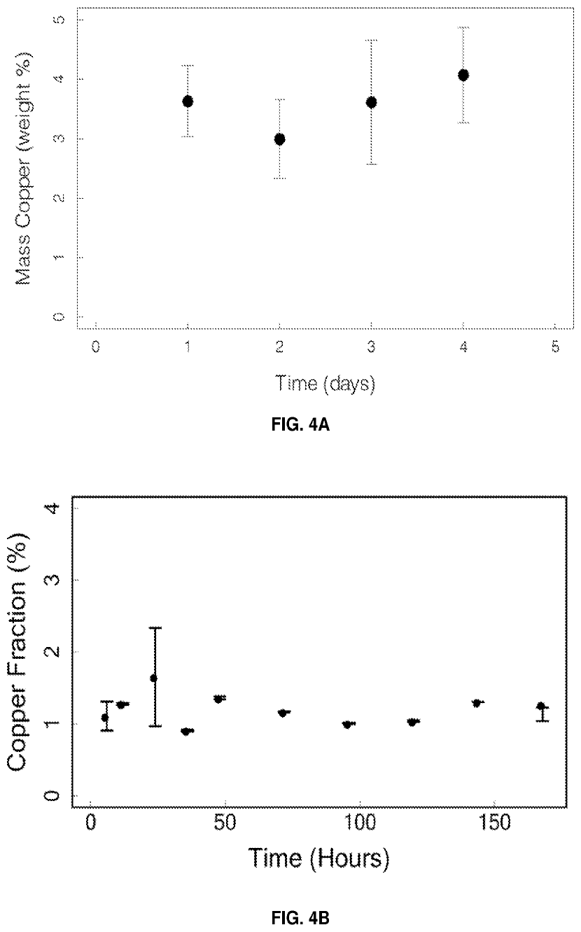

Cu-ZSM5 samples with varied ion exchange times (1-5 days) were analyzed to determine whether copper loading would increase with ion exchange time. As no significant trend was found (FIG. 4), 24 hours appears to be a sufficient ion exchange duration. Additionally, these copper loadings were comparable to those of Grudner, S., 2016, Narsimhan, K., 2016, and Ipek, B., 2016 implying that that this process can be completed successfully without the need for heat, or successive ion exchanges. See, for example, S. Grundner, et al. Synthesis of single-site copper catalysts for methane partial oxidation Chem. Commun., 2016, 52, 2553, which is incorporated by reference in its entirety. While there certainly could be improvements made to copper loading; determining unnecessary procedural steps or resources is essential when considering potential production at scale.

### X-Ray Diffraction

XRD was utilized to determine if a well-defined active site motif with repeating crystal structure was present in substantial quantities within samples of activated Cu-ZSM5 catalyst. While there are many proposed active site motifs in the literature, it is poorly agreed upon which may be the most advantageous or likely to occur. See, for example, Ambarish R. Kulkarni, et al., Cation-exchanged zeolites for the selective oxidation of methane to methanol Catal. Sci. Technol., 2018; Karthik Narsimhan, et al. Catalytic Oxidation of Methane into Methanol over Copper-Exchanged Zeolites with Oxygen at Low Temperature ACS Cent. Sci. 2016, 2, 424-429; Yayun Shi, et al. (2020) Quasicatalytic and catalytic selective oxidation of methane to methanol over solid materials: a review on the roles of water, Catalysis Reviews, 62:3, 313-345; and Allegra A. Latimer, et al., Direct Methane to Methanol: The Selectivity-Conversion Limit and Design Strategies ACS Catal. 2018, 8, 6894-6907, each of which is incorporated by reference in its entirety.

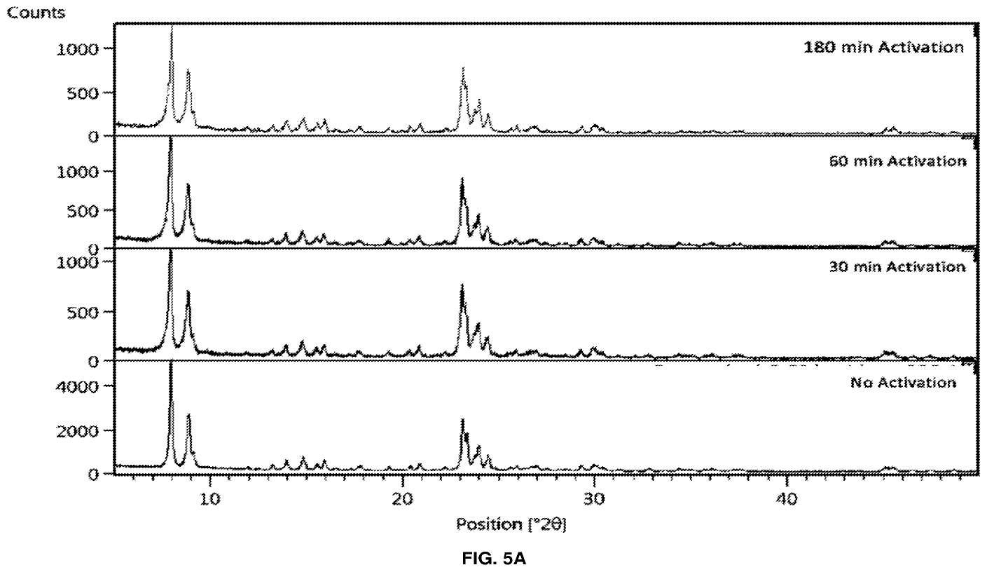

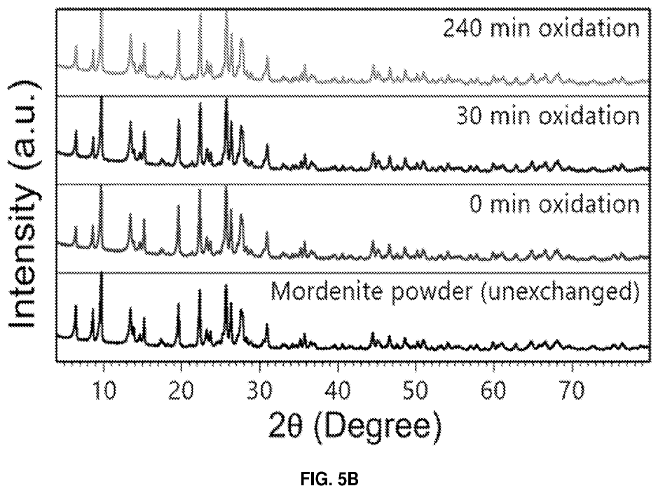

XRD samples were prepared over a range of activation times (0 to 180 minutes See FIGS. 5A-5B. Previous studies included activation times (between 2 and 24 hours). See, for example, Ambarish R. Kulkarni, et al., Cation-exchanged zeolites for the selective oxidation of methane to methanol Catal. Sci. Technol., 2018; Kimberly T. Dinh, et al., Continuous Partial Oxidation of Methane to Methanol Catalyzed by Diffusion-Paired Copper Dimers in Copper-Exchanged Zeolites J. Am. Chem. Soc. 2019, 141, 11641-11650; B. Ipek and R. F. Lobo Catalytic conversion of methane to methanol on Cu-SSZ-13 using N₂O as oxidant Chem. Commun., 2016, 52, 13401; Karthik Narsimhan, et al. Catalytic Oxidation of Methane into Methanol over Copper-Exchanged Zeolites with Oxygen at Low Temperature ACS Cent. Sci. 2016, 2, 424-429; Yayun Shi, et al. (2020) Quasicatalytic and catalytic selective oxidation of methane to methanol over solid materials: a review on the roles of water, Catalysis Reviews, 62:3, 313-345; Allegra A. Latimer, et al., Direct Methane to Methanol: The Selectivity-Conversion Limit and Design Strategies ACS Catal. 2018, 8, 6894-6907; and S. Grundner, et al. Synthesis of single-site copper catalysts for methane partial oxidation Chem. Commun., 2016, 52, 2553, each of which is incorporated by reference in its entirety.

### Results

A biomimetic copper zeolite capable of converting atmospheric- and low-level methane at relatively low temperatures (e.g., 200-300° C.) in simulated air. Depending on the duty cycle, 40%, over 60%, or complete conversion could be achieved (via a two-step process at 450° C. activation and 200° C. reaction or a short and long activation under isothermal 310° C. conditions, respectively). Improved performance at longer activation was attributed to active site evolution, as determined by X-ray diffraction.

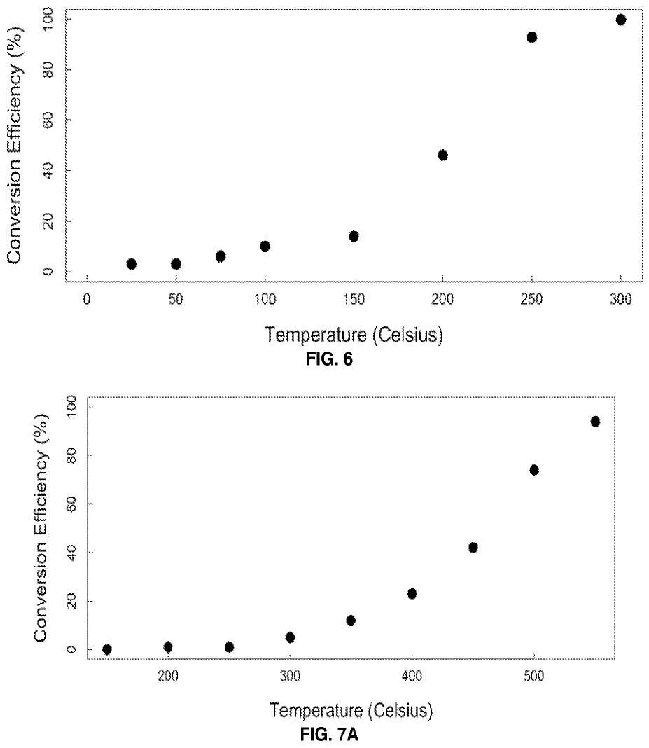

In order to evaluate the catalysts conversion efficiency at low temperatures the reaction was carried between 25 to 350° C. Analysis of copper zeolites considered low temperature are typically reported between 150 and 200° C. See, for example, Ambarish R. Kulkarni, et al., Cation-exchanged zeolites for the selective oxidation of methane to methanol Catal. Sci. Technol., 2018; Kimberly T. Dinh, et al., Continuous Partial Oxidation of Methane to Methanol Catalyzed by Diffusion-Paired Copper Dimers in Copper-Exchanged Zeolites J. Am. Chem. Soc. 2019, 141, 11641-11650; B. Ipek and R. F. Lobo Catalytic conversion of methane to methanol on Cu-SSZ-13 using N2O as oxidant Chem. Commun., 2016, 52, 13401; Karthik Narsimhan, et al. Catalytic Oxidation of Methane into Methanol over Copper-Exchanged Zeolites with Oxygen at Low Temperature ACS Cent. Sci. 2016, 2, 424-429; Yayun Shi, et al. (2020) Quasicatalytic and catalytic selective oxidation of methane to methanol over solid materials: a review on the roles of water, Catalysis Reviews, 62:3, 313-345; Allegra A. Latimer, et al., Direct Methane to Methanol: The Selectivity-Conversion Limit and Design Strategies ACS Catal. 2018, 8, 6894-6907; and S. Grundner, et al. Synthesis of single-site copper catalysts for methane partial oxidation Chem. Commun., 2016, 52, 2553, each of which is incorporated by reference in its entirety. In these experiments the activation temperature was kept constant at 450 C. Small amounts of methane were shown to oxidize at temperatures as low as 50-75° C. and reached complete conversion by 300° C. (FIG. 6), confirming the strong potential of copper zeolites as methane sequestration catalysts. Moreover, at 200° C., the methane effluent concentration was reduced by nearly 46%, nearly two orders of magnitude improvement over existing selectivity optimization studies. See, for example, Ambarish R. Kulkarni, et al., Cation-exchanged zeolites for the selective oxidation of methane to methanol Catal. Sci. Technol., 2018; Kimberly T. Dinh, et al., Continuous Partial Oxidation of Methane to Methanol Catalyzed by Diffusion-Paired Copper Dimers in Copper-Exchanged Zeolites J. Am. Chem. Soc. 2019, 141, 11641-11650; B. Ipek and R. F. Lobo Catalytic conversion of methane to methanol on Cu-SSZ-13 using N2O as oxidant Chem. Commun., 2016, 52, 13401; Karthik Narsimhan, et al. Catalytic Oxidation of Methane into Methanol over Copper-Exchanged Zeolites with Oxygen at Low Temperature ACS Cent. Sci. 2016, 2, 424-429; Yayun Shi, et al. (2020) Quasicatalytic and catalytic selective oxidation of methane to methanol over solid materials: a review on the roles of water, Catalysis Reviews, 62:3, 313-345; and Allegra A. Latimer, et al., Direct Methane to Methanol: The Selectivity-Conversion Limit and Design Strategies ACS Catal. 2018, 8, 6894-6907, each of which is incorporated by reference in its entirety. This value was used to establish a 200° C. reaction temperature for the remaining experiments, to allow for correlation of changes in conversion efficiency with changes in activation parameters.

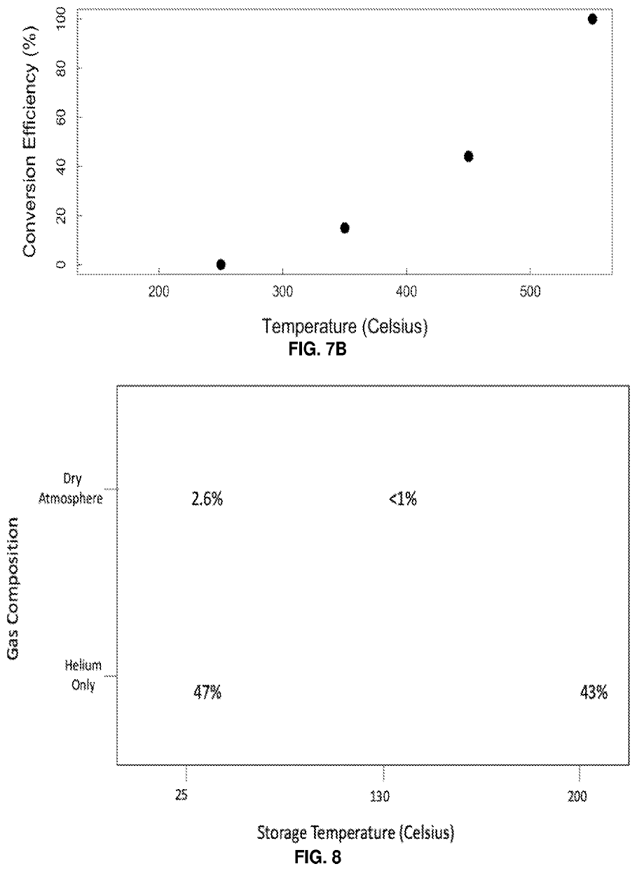

In order to assess the viability of this catalyst as a sustainable solution for methane oxidation, it is important to minimize energy use during synthesis. The catalyst activation step was performed at a range of temperatures and catalytic efficiency was analyzed after a subsequent reaction step at 200° C. Limited catalyst activation was achieved as low as 300° C. (FIG. 7A), lower than has previously been attempted with the exception of scenarios which were assisted with high pressures. See, for example, Patrick Tomkins, et al. Isothermal Cyclic Conversion of Methane into Methanol over Copper-Exchanged Zeolite at Low Temperature Angew. Chem. Int. Ed. 2016, 55, 5467-5471, which is incorporated by reference in its entirety. Samples were also oxidized with 20% oxygen balance helium (FIG. 7B), and performed equally as well at each temperature. While oxidation of metal zeolite catalyst has been explored using nitrous oxide or in aqueous solution using hydrogen peroxide, the use of oxygen at low concentrations for this purpose is novel. See, for example, Kimberly T. Dinh, et al. Continuous Partial Oxidation of Methane to Methanol Catalyzed by Diffusion-Paired Copper Dimers in Copper-Exchanged Zeolites J. Am. Chem. Soc. 2019, 141, 11641-11650; Yayun Shi, et al. (2020) Quasicatalytic and catalytic selective oxidation of methane to methanol over solid materials: a review on the roles of water, Catalysis Reviews, 62:3, 313-345; and Allegra A. Latimer, et al. Direct Methane to Methanol: The Selectivity-Conversion Limit and Design Strategies ACS Catal. 2018, 8, 6894-6907, each of which is incorporated by reference in its entirety. Its success under these conditions is promising for the potential use of CuMOR as a sustainable solution.

If catalyst activation is achievable at temperatures as low as 300 C in 20% oxygen, it is possible that the production process could be performed in large quantities at an industrial scale without the need for a gas-controlled environment. To address this, activated catalyst was stored for a duration of at least one hour before the reaction step and efficiency analysis (FIG. 8). While the catalyst continued to perform effectively after storage at low temperature, before reheating to 200° C. for reaction, samples that were removed from the reactor for storage no longer maintained their catalytic abilities. All samples removed from the reactor were stored in dry environments. Further investigation is necessary to determine the source of the deactivation.

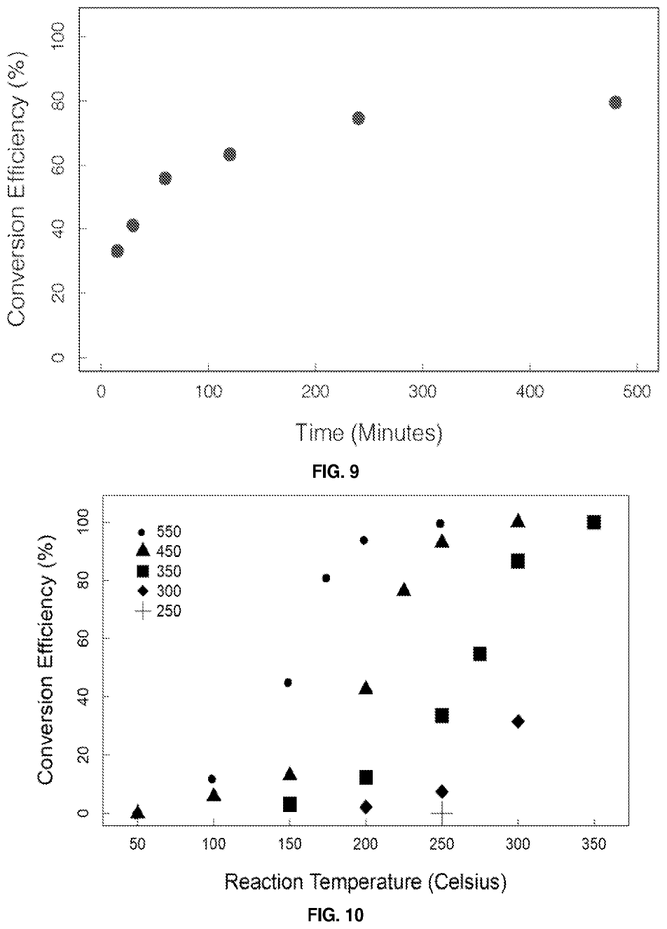

Referring to FIG. 9, a graph depicting conversion efficiency and activation time shows that conversion efficiency can be optimized at 250 minutes under conditions of 450° C., 20% O₂ activation and a conversion reaction at 200° C. and 2 ppm methane in 20% O₂/80% He.

Referring to FIG. 10, a graph depicting conversion efficiency and conversion reaction temperature at different activation temperatures, which shows that 50% conversion efficiency can be achieved at temperatures between 150 and 200° C., when activated at 450 to 550° C. The conditions for these experiments were 20% O₂ activation for 30 minutes and conversion reactions of 2 ppm methane in 20% O₂/80% He.

Referring to FIG. 16, a graph depicting conversion efficiency and multiple reactivations shows carbon conversion efficiency.

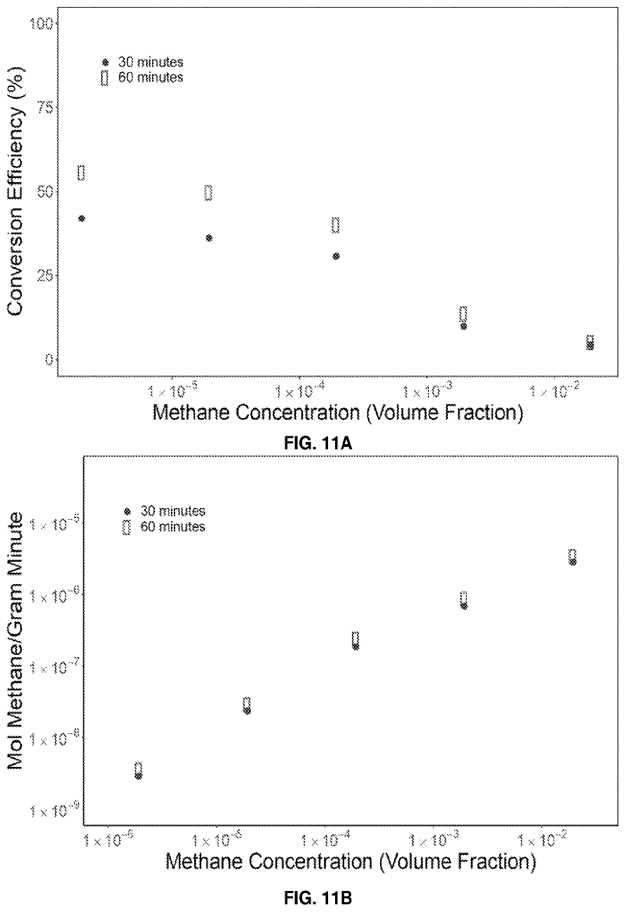

Referring to FIGS. 11A-11B, conversion efficiency over a range of inputs up to 2% methane is demonstrated.

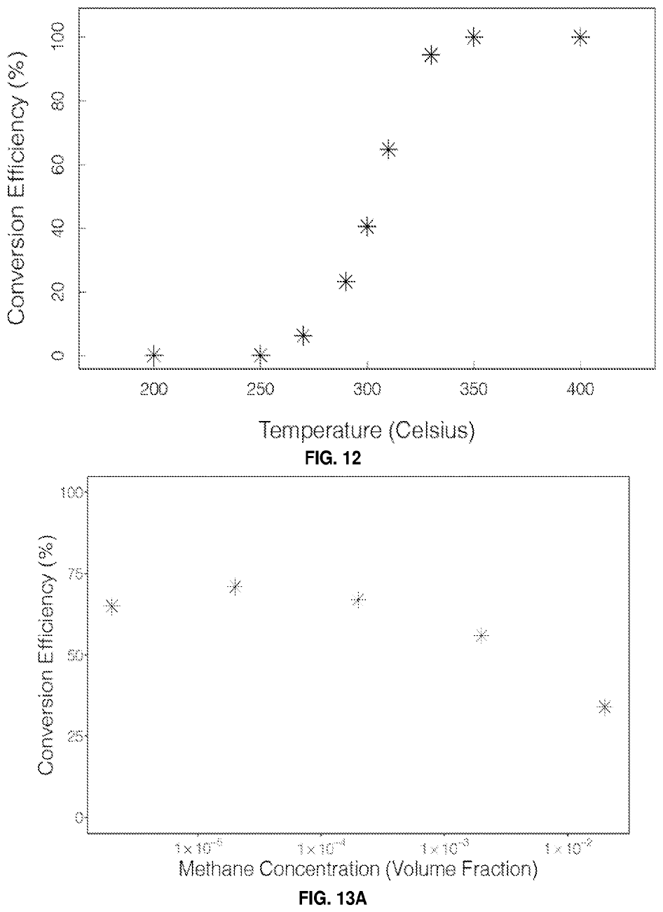

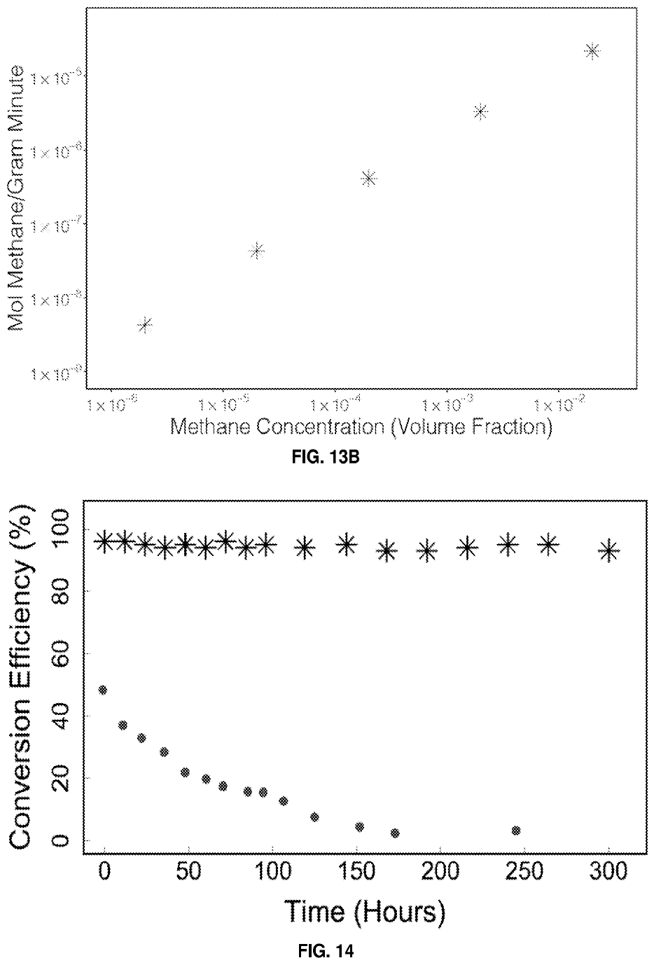

Isothermal runs have been investigated. FIG. 12 is a graph depicting conversion efficiency over a range of temperatures, which shows conversion efficiency increases significantly at temperatures between 250° C. and 350° C., particularly at temperatures greater than 300° C. and less than 350° C. For example, when activated for 30 minutes at 310° C. in 20% O₂ without methane and conversion at 310° C. with methane in 20% O₂, efficiency was high at lower concentrations of methane. This surprising result is shown in FIGS. 13A-13B are graphs depicting conversion efficiency and production over a range of methane concentrations. Referring to FIG. 14, the stability of this isothermal system at 310° C. is shown, with little change in conversion efficiency after 300 hours. In this experiment, the catalyst was activated for 8 hours. The experiment was terminated at 12.5 days without an indication of significant degradation of performance.

### Discussion

The results of this study demonstrate high methane conversions efficiencies at low temperatures with a minimally intensive synthesis procedure. To date, copper zeolite catalysts have been characterized in the literature with methane conversion efficiency ranges below 1%. Here 100% conversion and catalytic activity as low as 75° C. was achieved. Furthermore, the catalyst was activated at lower temperatures with lower oxygen concentrations than previously explored. The synthesis procedure outlined here was performed under the coolest, shortest, and least technically precise conditions described in the literature. See, for example, Ambarish R. Kulkarni, et al., Cation-exchanged zeolites for the selective oxidation of methane to methanol Catal. Sci. Technol., 2018; Kimberly T. Dinh, et al., Continuous Partial Oxidation of Methane to Methanol Catalyzed by Diffusion-Paired Copper Dimers in Copper-Exchanged Zeolites J. Am. Chem. Soc. 2019, 141, 11641-11650; B. Ipek and R. F. Lobo Catalytic conversion of methane to methanol on Cu-SSZ-13 using N2O as oxidant Chem. Commun., 2016, 52, 13401; Karthik Narsimhan, et al. Catalytic Oxidation of Methane into Methanol over Copper-Exchanged Zeolites with Oxygen at Low Temperature ACS Cent. Sci. 2016, 2, 424-429; Yayun Shi, et al. (2020) Quasicatalytic and catalytic selective oxidation of methane to methanol over solid materials: a review on the roles of water, Catalysis Reviews, 62:3, 313-345; Allegra A. Latimer, et al., Direct Methane to Methanol: The Selectivity-Conversion Limit and Design Strategies ACS Catal. 2018, 8, 6894-6907; and S. Grundner, et al. Synthesis of single-site copper catalysts for methane partial oxidation Chem. Commun., 2016, 52, 2553, each of which is incorporated by reference in its entirety. All of these factors build toward the promise of a viable methane sequestration technology.

### Methods

### Copper Mordenite Synthesis

Ammonium mordenite zeolite powder (5±0.1 g; Alpha Aesar) was stirred with 0.05 M copper nitrate solution (500 mL) for 22-26 h and then vacuum-filtered through a glass fiber filter (0.7 ism GFF). Filtered solids were dried at 130° C. for 10-14 h, transferred to a glass vial, and stored in a desiccator until use. Importantly, it is noted that this preparation route is benign and strives to meet Green Chemistry principles: the ion exchange occurs at room temperature with minimized volumes and relies on earth-abundant, non-toxic materials, without acidic or organic solvents, with low energy requirements, and without the need for exotic or complex, multi-step syntheses. The process was highly reproducible.

### Reactor Design and Analytical Measurement

Gases were pre-mixed using a custom-built mass flow control array with electronic control, customized for the delivery of trace gases, including ultra-high-purity (UHP) helium, UHP oxygen, and 25, 700, and 70,000 ppmv methane in helium (Airgas). These were delivered to a vertically oriented, quartz tube furnace [16×½ O.D. inch (length×diameter)] fitted with a quartz frit and placed inside an Applied Systems 3210 series vertical tube furnace, which provided thermal control via an 850 W power supply. The reactor effluent was delivered to an SRI Instruments 8160C gas chromatograph with a flame ionization detector by direct injection every 90 s via two calibrated loops (5 mL each) and a Valco Instruments eight-port valve. The GC was calibrated daily with authentic standards (Mesa Specialty Gases).

### Catalyst Activation and Methane Conversion Reactions

In all cases, after loading 1 g of copper mordenite (approximately 3 cm height) into the vertical tube furnace, the reactor volume (34.5 mL) was flushed with helium while heating prior to introduction of activation gases. Then, experiments were conducted in one of two modes: a two-step activation-then-reaction or an isothermal reaction. For the “two-step” process, the catalyst was activated in an atmosphere of 20% oxygen and 80% helium (at 450° C. for 30 min unless otherwise noted) or 100% oxygen. Subsequently, the conversion reaction was carried out in the same atmosphere with additional methane (0.0002-2% methane; 30 min at 200° C. unless otherwise indicated). Note that traditional work on these types of materials for methane-to-methanol conversion relies on the two-step process to avoid overoxidation of methane to CO₂. In the activation step, 100% O₂ atmospheres are used to maximize efficacy, precluding simultaneous delivery of methane and necessitating segregated activation and reaction steps. In the experiments, the operation in real air (e.g., 20% O₂ with methane) was simulated. In all cases, the total flow of gas constant was held to preserve an approximately 30 s residence time of gas in the reactor. For the “isothermal” process, the catalyst was activated for 30 min (or 8 h for a long-duration study) under methane-free, 20% oxygen, and 80% helium and then reacted in 0.0002% methane for 30 min (or 300 h for a long-duration study) at a constant temperature. In order to achieve a constant temperature in the reactor, a continuous supply of heat was provided through a power supply with temperature feedback. Isothermal processes were explored to investigate the possibility of catalytic function without a thermal cycle (i.e., duty cycle), which brings a net energy and operational expense savings during operation (e.g., energy savings are conferred by avoiding heating and cooling cycles).

At the beginning of the reaction step, directly following the introduction of methane to the feed stream (70 sccm in total), a 15 min flushing time was allowed to ensure adequate mixing within the catalyst bed volume. After this equilibration phase, injections were made every 90 s for an additional 30 min (21 injections), and an average of these values (21 injections for the two-step reactions; five injections for isothermal reactions) was used to establish the effluent concentration. Standard deviations were less than 2.6% of the mean. Conversion efficiency was calculated by comparing the effluent methane concentration with the influent methane concentration (no catalysts, same gas mixture) at the same temperature. There was no evidence of non-catalytic methane oxidation (i.e., direct combustion) at any temperature below 550° C., and copper-free mordenite zeolite powder illustrated no potential to convert methane at the assayed temperatures (i.e., 200° C.).

### Material Characterization

Copper-doped mordenite metal content was determined by inductively coupled plasma mass spectrometry (ICP-MS) using a NexION 300D. Briefly, samples were prepared by refluxing approximately 1 g of catalyst in 50 mL of 50% v/v HNO₃ for 2 h until the solids were visibly bleached white. The leachate was extracted, concentrated to 10% of its initial volume, and then reconstituted in 2% HNO₃. All copper loadings fell between 1 and 2 weight percent. Copper contents of ion exchanged zeolite (Mordenite) were determined using inductively coupled plasma mass spectrometry (ICP-MS). Copper zeolite powder (0.24-0.26 g) was leached in 20 mL of 100% v/v nitric acid over a hotplate and refluxed for 1 hour. At this point, the solution was uncovered and allowed to vaporize until less approximately 1 mL of liquid remained. The remaining liquid filtered with a 0.22-micron filter, and reconstituted to 50 mL with a 2% nitric acid solution. Solutions were spiked with a multiple component internal standard (Multi-element Calibration Standard 3 by Perkin Elmer). Five-point calibration standards were made with a 1,000 ppm copper standard in 2% nitric acid. Samples of copper zeolite were collected after ion exchange times varying from 0.25 to 7 days and the copper loadings reached equilibrium after 6 hours. See FIG. 4B.

The crystal structure of the copper zeolite was studied by X-ray diffraction (XRD) using a PANalytical X'pert PRO diffractometer equipped with Bragg-Brentano geometry and Ni-filtered Cu Kα radiation (λ=1.5418 Å, 45 kV, 40 mA); data were recorded in the range of 5-80 2θ. All results were consistent with the dominant crystal structure of the native zeolite. Scanning electron microscopy was conducted using a Zeiss Merlin Gemini field emission scanning electron microscope (5 kV accelerating voltage; 184 pA probe current). A photograph of the catalyst before and after use shows a noticeable color change.

### Activation and Reuse Potential

The performance efficiency of catalysts for methane capture and conversion is typically evaluated under a two-step process, where the first step (“activation”) occurs in methane-free, oxygen-rich conditions, and the second step (“reaction”) occurs in the presence of the reagent methane. Conducting all reaction steps at 200° C. allowed the evaluation of the activation step parameters spanning a range of thermal, temporal, and gas composition conditions. After relatively short (30 min) activations, increasing temperature in 100% oxygen improved reaction conversion efficiencies, where modest conversion efficiencies of 40% were observed at 450° C., and 80-100% conversion was shown at 500 and 550° C., respectively (FIGS. 7A-7B). These same efficiencies were observed in 20% oxygen atmospheres, indicating that the oxygen level was not limiting to the activation, and thermal effects may be more important. This has promising implications for field deployment of the catalyst as ambient oxygen levels (e.g., from air) might be useful for catalyst activation, rather than requiring the presence of explosive levels of reactant oxygen. While the high end of observed activation temperatures and durations are within the range of previous studies (at or above 450° C. and with durations at or above 1 h), use of ambient levels of oxygen has not been demonstrated previously for copper zeolites. Some novel “lean methane” catalysts based on cobalt, nickel mixtures, or platinum group metal (PGM) structures have been shown to work at or below 10% oxygen, but these require both high temperatures, expensive or complex synthetic processes, applied voltage, and/or a combination of activation gases far from ambient.

The impact of activation time was clear, where progressively longer 450° C. treatments conferred better methane conversion efficiency during the reaction. A short, 15 min activation resulted in only modest conversion, but this increased to over 60% by 2 h and nearly 80% after 8 h. While the conversion metrics of previous studies are not directly comparable, prior methane-to-methanol conversion attempts typically utilize oxidations of 1 h or longer. Extremely short or implicit activation times in a unique reaction environment have shown little reactivity (e.g., heating to 270° C. in 1% oxygen, 3% water, and 18% methane; 0.03% conversion). In this work, because copper oxidation is kinetically fast, the time dependence suggests (1) that either gas transport through the constrained zeolite pore structure is slow or (2) that there is a critical, thermally mediated structural rearrangement of the copper zeolite or atoms therein that occurs slowly. While the former could be addressed by constructing hierarchical materials with improved gas access without compromising the pore structure, the latter might require re-engineering of the catalyst nano-structure or strategic thermal pre-treatments as part of the catalyst preparation.

To explore the possibility that longer-duration thermal pretreatments were giving rise to uniquely active catalyst nano- or mesostructures, powder XRD was utilized to probe the existence of a thermally driven catalyst rearrangement (FIGS. 5A-5B). As a whole, the XRD spectra were consistent with mordenite powder structures, and no quantifiable copper or copper oxide phase was observed (likely due to the low loading of Cu; ca. 1%). As thermal treatment time increased, deformations in peak patterns emerged at low diffraction angles (less than 10° 2θ). These peaks correspond to planes that bisect mordenite's large channel (6.5×7 Å) pores lengthwise (110, 020, and 200), whereas smaller pocket pores (2.6×5.7 Å) were not affected. Specific evolutions found were that the integral breadth of the first and second observable peaks (corresponding to the 110 and 020 planes, respectively) increased and decreased in equal measure (approximately 0.01 rad. Note that the integral breadth (area of the peak divided by height) was employed because it is less susceptible to over interpretations of changes in peak shape at low angles that would be associated with the extractable parameter full width at half-maximum). As integral breadth is inversely proportional to the average crystallite thickness normal to the reflecting plane, these observations are consistent with a crystal structure development in the mordenite pore channels over time, where the channel became more compressed normal to the 110 plane and more expanded normal to the 020 plane with longer duration thermal treatments. Thus, the longer activation times appear to be associated with a micro-structural change in the larger pore structure, which leads to better methane conversion.

Such long in situ activation timescales would only be practical in field deployments if the catalyst could be recycled or activated and then reacted in series. Engineered strategies to achieve this type of cycling chemistry exist in the form of reactors that can be strategically cycled (in series or parallel), such as regenerative thermal oxidation, regenerative catalytic oxidation, or catalytic recuperative oxidizers, all developed for the conversion of volatile organic compounds (VOCs) in industrial waste streams. With this in mind, catalyst re-activation was illustrated for at least six cycles, showing a general trend of improvement consistent with the effect of longer activation times. This repeatability suggests that the total methane conversion potential per unit of catalyst may be sufficiently high to reduce the cost of CO₂ equivalent capture below critical thresholds of 15-50 USD/ton. Importantly, these reuse experiments were conducted by oscillating between 450° C. activation in 100% oxygen and 200° C. reaction in 20% oxygen, which would necessitate undesirable life cycle costs associated with the duty cycle and risk associated with the use of concentrated oxygen. As such, a broader range of reaction conditions and operation strategies was explored.

### Methane Conversion Approach

Minimizing the temperature requirements of low-to-ambient level methane conversion reduces the energetic demand and infrastructural requirements of reactors, improves the overall operational lifecycle greenhouse gas benefit, and expands the possible deployment opportunities by reducing the flammability or explosion hazard (e.g., for ventilation air at coalmines). To evaluate the potential for use at low temperatures, conversion reaction temperature from 50 to 350° C. was varied over a range of activation temperatures between 250 and 550° C. At high activation temperatures (above 450° C.), non-zero catalytic activity was achieved at reaction temperatures as low as 100° C., and over 40% conversion was achieved at 200° C. and above. At lower activation temperatures (350° C. or below), conversion over 40% requires systematically higher reaction temperatures (above 250° C.). Limited but quantifiable conversion was observed at temperatures as low as 100° C. (FIG. 10), reduced from previous experiments carried out between 150 and 200° C. Catalytic conversion of methane using PGMs, cobalt or nickel-cobalt mixtures has typically relied on higher activation temperatures (500-1000° C.) or long durations (e.g., 24 h at 350-400° C.); that is, successful activations generally exceed the time and temperature ranges explored here. Using relatively aggressive activations, successful conversions of 1000-10,000 ppmv methane (0.1-1% CH₄, with 10% or less oxygen) at temperatures well below the ignition point (600° C.) have been recorded with a few demonstrating conversion temperatures as low as 300° C. While the reaction temperatures that were observed are moderately lower than previous demonstrations, the dramatically different activation procedures and simplified catalyst synthesis offer unique benefits. As a point of comparison, conversion efficiencies to CO₂ are not often reported and cannot be directly compared to the results; nevertheless, methanol production rates in previous studies have been quite low (order 0.1-0.3 mol methanol per mol Cu or less; methanol production was not systematically quantified in this study, but early spot checks in the two-step process revealed around 1-2 orders of magnitude lower methanol in the post-reaction, water-extracted catalyst, where the input methane was around 106-fold lower than in previous studies). While CO₂, formaldehyde, formic acid, and methanol are the only anticipated products in the oxidation pathway, the potential formation of other products to date was not evaluated. It appears that no prior studies have shown complete conversion of methane at these temperatures in simulated air with an easy-to-produce, earth-abundant catalyst.

The achievement of harmonized catalyst activation and reaction temperatures confers important operational and lifecycle advantages. Specifically, the isothermal operation minimizes the need to repeatedly deliver power for heating the thermal mass of a reactor and the associated catalyst, reducing the levelized cost, energy, and greenhouse emissions. Using the operational space as a roadmap (FIG. 10) for optimizing catalytic efficiency, the isothermal reaction offers complete removal of atmospheric methane at 350° C. (FIG. 12) was demonstrated. Minor conversion (approx. 7.1%) was initially detected at 270° C. This is consistent with the novel continuous reactions conducted by Dinh et al., who demonstrated methanol production at this temperature, albeit with efficiencies below 1% (by design, to minimize “over-oxidation” to CO₂ and in dramatically different conditions). At modestly higher temperatures (e.g., 300-310° C.), 42-67% of methane was oxidized following a 30 min activation. This is valuable for some remediation applications and could be pushed to higher conversion rates under different activation schema (see below). Levelized life cycle budgets that consider the potential trade-off between total methane abatement versus operational energy demands (both in CO₂ equivalents) must be conducted to determine if the additional conversion merits the operational temperature. Theoretically speaking, the conversion of methane to CO₂ is exothermic, and depending on the reactor design and flowrates, higher input concentrations of methane might be able to generate excess heat. For example, ventilation air in mines can contain between 0.1 and 2% methane. A simple calculation of energy generation compared to energy requirements can be derived from the theoretical energy generated by the reaction relative to the energy needed to heat incoming air to the operating temperature (eq 1).

$${m}_{{CH}_{4}} Δ {H}_{rxn} / {m}_{air} {C}_{p} Δ {T}_{air} \qquad ( 1 )$$

where m_{CH4} is the mass of methane in the incoming air stream, ΔHᵣₓₙ is the enthalpy of methane oxidation to CO₂ (890 KJ/mol), mair is the mass of incoming air with specific heat, Cp (700 J/kg K), and ΔTair is the temperature change required to get from the ambient temperature to the operating temperature (e.g., 310° C.). At these sources, a methane concentration of 1% gives a heat-generation-to-heat-demand ratio of 1.5; that is, the process generates excess energy. At the ventilation air flowrates used at mines (approximately 100,000-1,000,000 CFM), this could potentially yield electricity at the power-plant scale (order 5 KW, depending on specifics of the system). One could use this excess energy to pre-heat incoming air, offset the energy demands of the system (e.g., “parasitic power” associated with air movement across a pressure drop), and ultimately drive electricity-generating heat exchangers to meet other needs on-site. Notably, such a system could accelerate payback times for the device as well. Considering that low-level methane sources responsible for the majority of emissions to the atmosphere span 4 orders of magnitude in concentration, itis possible and somewhat remarkable that this catalyst could be deployed with a net energy yield in modestly elevated methane scenarios (over approximately 0.67% methane).

### Methane Abatement at All Sub-Flammable Thresholds

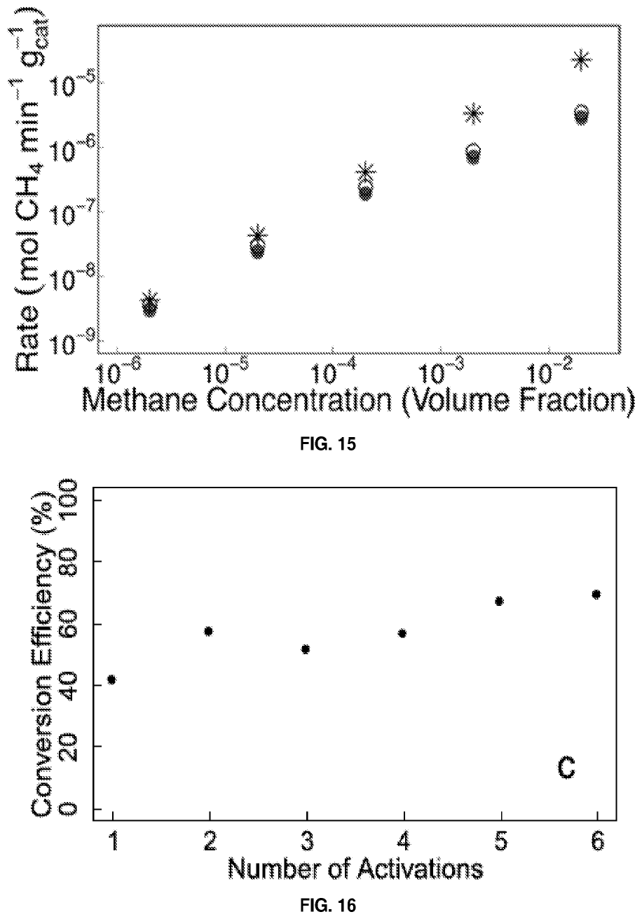

To explore the possibility of deploying the catalyst for methane abatement at any sub-flammable level, incoming methane conversion rates were evaluated between 2 ppmv and 2% v/v methane (e.g., from near-atmospheric to typical ventilation air levels observed in coal mines). Overall, higher incoming methane corresponded with higher rates of conversion (FIG. 15; 4.28×10⁻⁹ to 2.28×10⁻⁵ mol min⁻¹ g_{catalyst}⁻¹ isothermally) but came at a cost to the total proportion of methane removed. Further, the conversion rate exhibited sensitivity to the activation time and temperature: isothermal reactions at 310° C. exhibited steady and monotonic increases in the methane conversion rate, whereas step-wise reactions at short and long (30 and 60 min at 450° C.) activations followed by lower temperature reactions (200° C.) showed a diminishing conversion rate with higher methane loadings. The influence of thermal history (i.e., duration of elevated temperature treatment in both continuous and non-continuous modes) implies that there is a physical limitation to catalyst activation, consistent with the earlier observation of the importance of activation time. Promisingly, there does not appear to be a definitive ceiling in the catalyst's ability to convert methane at higher concentrations, implying that higher conversion rates could be achieved via optimization of activation parameters and/or reactor geometry. Hierarchical material construction, such as preserving the nanoconfinement of the Cu-aluminosilicate active sites but supporting those on a substrate to promote better gas-catalyst contact, improved heat transfer properties by varying the material choice, and novel reactor design are all viable routes to enhance conversion rates. This potential for improvement aside, for the first time, these results demonstrate that copper mordenite can convert methane at low-level concentrations previously untested by either other zeolites, lean combustion, or ventilation air methane catalysts. Considering that these conversions can be achieved at any methane concentration of concern well below flammability limits (less than 5%) or ignition temperatures, strategic deployment at or near elevated methane sources would bring rapid benefit to reduce or reverse the exponential accumulation of atmospheric methane observed since the early 1800s.

### Catalyst Conversion Capacity and Lifetime

Viability and adoption of this catalytic technology will depend on both the opportunity for levelized greenhouse gas reductions and the total cost of the abatement strategy. Both scale with the reusability of the catalysts and requisite strategy for regeneration (e.g., thermal cycling or chemical recharge). First, a traditional two-step activation followed by a reaction at differential temperatures illustrated a relatively rapid deterioration in the methane conversion potential (30 min, 450° C. activation in 20% oxygen followed by continuous exposure to 200° C., with 2 ppmv methane added; FIG. 14). In contrast, re-activation for 8 h followed by isothermal reaction at 310° C. showed prolonged, near complete methane removal for up to 300 h (12 days). The high reactivity initially was a consequence of the long activation time and consistent with earlier observations (FIG. 9) that activation time promotes formation of reactive pore structures observed by XRD (FIGS. 5A-5B). Maintenance of a strong catalytic activity over this time suggested that either the catalyst capacity was never reached or that the catalyst was reactivating and performing in a continuous manner (i.e., regenerating itself, as in the definition of a catalyst). The dramatically different behavior observed between a two-step activation and an isothermal operation is the outcome of both thermal and kinetic effects. In a two-step process, the short, high-temperature (450° C.) activation is followed by operation at a relatively low temperature (200° C.). The continuous decay in performance implies that 200° C. is insufficient to reactivate and fails to maintain the activity of the catalyst. The low conversion efficiencies (approaching 0% methane conversion) are consistent with low-temperature activation experiments (see FIG. 10 for 250° C. activation and reaction). In contrast, a long activation step (8 h) at 310° C. gave a high and consistent methane conversion performance. The near complete conversion of methane (100%) exceeded the conversion at 300 or 350° C. for only 30 min (40-50% methane conversion; FIG. 10), reinforcing the sensitivity of catalyst performance to activation time (see FIG. 9) associated with catalyst pore re-structuring (FIGS. 5A-5B). While the long-duration, 8 h activation could initially produce a very reactive catalyst structure that is maintained or regenerated, itis also possible that the isothermal reaction condition is functionally serving as a very long “activation” in and of itself, conferring sustained catalytic activity.

Continuous reactivity under isothermal operations increased the overall quantity of converted methane more than fivefold compared to the two-step reaction (0.3 vs 1.7 mg CH₄ g_{catalyst}⁻¹ total when integrating over the duration of the long-term trials). Here, it was noted that these conversion ratios are only relative to one another and cannot be used to determine the total potential of the catalyst for methane drawdown from the atmosphere or near more concentrated sources. Indeed, evidence suggests that the total conversion capacity may be much higher if larger input methane concentrations were delivered (FIG. 15).

### Implications, Novelty, and Feasibility

The application of zeolites for biomimetic methane conversion has been explored for decades, but several key limitations existed in prior work that obfuscated the utility of these catalysts for low-level methane abatement. First, there had been a focus on stopping the reaction at methanol, so that methanol could be used as a chemical feedstock. However, technical challenges existed that prevented the arrest of the reaction at methanol (which proceeds exergonically to CO₂) and separation of the liquid stream from the gaseous input. Further, current methanol markets (75 MMT in 2018) would only abate about 37.5 MMT of methane, enough to reverse atmospheric accumulation but insufficient to offset major atmospheric source terms and restore atmospheric methane levels quickly. Second, methanol separation is only technically feasible at the most concentrated methane input streams but remains uneconomic due to the relatively low methanol yields. Methanol is often recovered and measured via water extraction of the catalyst, which would create major technological hurdles in and of itself. Third, in an effort to avoid “over oxidation” to CO₂, a practice emerged in which zeolites were cycled between oxygen-rich and methane-rich environments (i.e., the activation and reaction steps, respectively). Some key surprising results of this work is (1) the demonstration that catalyst turnover can be achieved in air at modestly low temperatures, in a continuous fashion, and at exceedingly low levels of methane in the sub-flammable region and (2) the recognition CO₂ is the desired product, overcoming separation challenges while conferring major climate-impact reduction. Any concern for CO₂ generation as a pollutant is misguided: methane-to-CO₂ conversion brings an instantaneous radiative forcing reduction and converting 60% of the atmospheric methane reservoir (i.e., restoration to pre-industrial levels) would result in a modest 1.1 ppmv increase in CO₂ (e.g., from 415 ppmv CO₂ today to 416 ppmv CO₂).

The ability to mitigate low-to-ambient levels of methane in simulated atmosphere indicates promise to deploy a stand-alone system anywhere where fugitive methane emissions exist. However, maximum environmental impact and economic yield (i.e., greenhouse gas (GHG) equivalents removed per $) would come with strategic deployment at relatively enriched methane sources (e.g., dairy and meat barns or coal mines). The potential for low or no energy needs on-site depending on the input methane concentration and the associated thermal yield was described. At high methane sources (e.g., coal and mineral mines, where ventilation air can be up to 2%) excess heat can meet the demands of heating incoming air and the catalyst and even offer excess electricity generation. This energy generation potential could improve the net GHG benefit of the technology. If modest carbon taxes are instated, the energy generation coupled with the sale of superfluous electricity could potentially pay back capital equipment expenses and operating costs over a relatively short time (order decade). Here, it is noted that the catalyst is synthesized from earth-abundant Cu and clay aluminosilicates. The copper-zeolite catalyst costs were estimated to be on the order of cents per pound—$0.15-0.82/1b, many orders of magnitude lower than those of competing technologies. Thus, there is great potential for this technology to be developed at low cost and with minimum environmental impact, potentially reducing operating costs below critical thresholds of proposed CO₂ pricing strategies (e.g., below $15-50/ton of CO₂ equivalents).

Several technical achievements remain on the path to feasible deployment. Catalyst poisoning regimes can be tested with typical atmospheric interferents (primarily water) and other light VOCs that might prematurely saturate or spoil the catalyst. Pre-filtration strategies may be needed to overcome any emergent complications, and real-world trans-formation products should be monitored on initial deployment. Ideally, control systems and in situ monitoring capability would ensure the continuous function and efficacy of any commercial device. Finally, the catalyst material should be supported or structured in a way such as to maximize air flow through the reactor system. Then, reactors could be interfaced downstream of extant air handling capacity on-site and further lower the levelized GHG impact and cost.

Other embodiments are within the scope of the following claims.

1. A method of oxidizing methane comprising:
activating a methane oxidation catalyst at an activation temperature at or below about 450° C. while exposed to an activation gas including less than 100% oxygen for an activation time; and
exposing the activated methane oxidation catalyst to a reaction gas mixture including less than 100% methane at a temperature of, or less than about 350° C. in the presence of an oxidative agent including less than 100% oxygen to convert the methane to an oxidized product.

2. The method of claim 1, wherein the activation time is 240 minutes or less.

3.-4. (canceled)

5. The method of claim 1, wherein the activation time is 90 minutes or less.

6. The method of claim 1, wherein the activation gas includes less than 80% oxygen.

7.-8. (canceled)

9. The method of claim 1, wherein the activation gas includes about 20% oxygen.

10. The method of claim 1, wherein the activation gas includes an inert gas.

11. The method of claim 1, wherein the inert gas is helium, argon or nitrogen.

12. The method of claim 1, wherein the activation gas is air.

13. The method of claim 1, wherein the activation temperature is below about 450° C.

14.-16. (canceled)

17. The method of claim 1, wherein the activation temperature is below about 250° C.

18. The method of claim 1, wherein the oxidative agent includes a liquid, a solid, or a gas.

19. (canceled)

20. The method of claim 1, wherein the oxidative agent includes less than 40% oxygen.

21. (canceled)

22. The method of claim 1, wherein the oxidative agent includes about 20% oxygen.

23. The method of claim 1, wherein the reaction gas mixture includes less than 50% methane.

24.-29. (canceled)

30. The method of claim 1, wherein the reaction gas mixture includes less than 10 ppm methane.

31. The method of claim 1, wherein the methane oxidation catalyst includes iron, copper, or nickel.

32.-33. (canceled)

34. The method of claim 1, wherein the methane oxidation catalyst includes a metal organic framework, a zeolite or an aerogel.

35. The method of claim 1, wherein the methane oxidation catalyst includes a copper zeolite, an iron zeolite, or a nickel zeolite.

36.-37. (canceled)

38. A system for oxidizing methane comprising:
a housing including:
an activation region for a methane oxidation catalyst that is activated at an activation temperature at or below about 450° C. while exposed to an activation gas including less than 100% oxygen for an activation time; and
a conversion region for the activated methane oxidation catalyst to convert a reaction gas mixture including less than 100% methane to an oxidized product at a temperature of, or less than about 350° C. in the presence of an oxidative agent including less than 100% oxygen.

39.-46. (canceled)

47. A method of oxidizing methane comprising:
exposing a reaction gas mixture including less than 4% methane in the presence of an oxidative agent including less than 22% or about 20% oxygen to a copper doped zeolite to convert the methane to an oxidized product.

48. The method of claim 47, wherein the reaction gas mixture including less than 4% methane in the presence of the oxidative agent including less than 22% or about 20% oxygen is exposed to the copper doped zeolite at a temperature of, or less than about 350° C.

49. The method of claim 47, wherein the oxidative agent is less than less than 22% or about 20% oxygen in an inert gas.

50. The method of claim 47, wherein the oxidative agent is air.
